(19) United States
(12) Patent Application Publication
(10) Pub. No.: US 2006/0216762 A1
(43) Pub. Date: Sep. 28, 2006

(54) Extracting reagent for hydrophobic analyte in whole blood

(75) Inventors: Alexander Belenky, Bronx, NY (US); Laurie Ann Livshin, Ardsley, NY (US); Minas Barbarakis, Katonah, NY (US)

Correspondence Address: RODMAN RODMAN, 7 SOUTH BROADWAY, WHITE PLAINS, NY, 10601, US

(73) Assignee: Bayer HealthCare LLC, Tarrytown, NY (US)

(21) Appl. No.: 11/089,261

(22) Filed: Mar. 24, 2005

(51) Int. Cl. G01N 33/567

(52) U.S. Cl. 435/7.21

(57) ABSTRACT

The present invention utilizes an extracting reagent to achieve quantitative detection of hydrophobic analytes from a biological test sample. The hydrophobic analytes can include steroids, and drugs. The biological test sample can include serum, plasma, whole blood, urine and spinal fluid. The hydrophobic analyte is preferably the drug cyclosporine, which is used primarily as an immunosuppressant. The biological test sample is preferably whole blood. The extracting reagent comprises a zwitterionic detergent and saponin, and optionally, a viscosity additive such as sucrose or glycerin. The detection and analysis of the hydrophobic analyte is preferably performed in an automated immunoanalyzer.

### BACKGROUND OF THE INVENTION

The body relies upon a complex immune response system to distinguish self from non-self. The proper functioning of the immune system is vital for the long-term health of the body.

Deficient immune response can lead to the body's inability to protect itself from non-self matter. Excessive immune response can lead to the body's overreaction to what would otherwise be harmless matter.

At times, the body's immune system must be controlled in order to either augment a deficient response or suppress an excessive response. For example, when organs such as kidney, heart, heart-lung, bone marrow and liver are transplanted in humans, the body will often reject the transplanted tissue by a process referred to as allograft rejection.

In treating allograft rejection, the immune system is frequently suppressed in a controlled manner through drug therapy. Immunosuppresant drugs are carefully administered to transplant recipients to help prevent allograft rejection.

Cyclosporine is an immunosuppressive agent. It affects the immune system of the body in such a way as to condition the body not to reject a transplanted organ. It also decreases the ability of the body to resist infections.

Even though cyclosporine is a highly effective immunosuppressant drug, its use must be carefully managed because the effective dose range is narrow. Excessive dosage can result in serious side effects, such as renal dysfunction, hypertension, cardiovascular cramps, hirsutism, acne, tremor, convulsions, headache, gum hyperplasia, diarrhea, nausea, vomiting, hepatotoxicity, abdominal discomfort, paresthesia, flushing, leukopenia, lymphoma, sinusitis and gynecomastia which have been observed in kidney, heart or liver transplant patients undergoing cyclosporine treatment. Too little cyclosporine can lead to graft rejection.

For effective immunosuppressant activity, the patient blood concentration of cyclosporine measured 24 hours after administering the drug will be generally maintained in a therapeutic range of from about 100 nanograms per milliliter (ng/ml) to about 400 ng/ml, depending on variable individual factors that control metabolic activity, including transplant type, age, diet, body weight and individual sensitivity.

The management of cyclosporine dosage requires careful control of the level or amount of the drug present in the patient. Because the distribution and metabolism of cyclosporine varies greatly between patients, and because of the wide range and severity of adverse reactions, accurate monitoring of the drug level is considered essential.

Laboratory methods for detection of cyclosporine have been developed. These techniques typically involve mass spectroscopy (MS) high performance liquid chromatography (HPLC), radioimmunoassay (RIA) or fluorescence polarization immunoassay (FPIA). However, such techniques produce inconsistent results, due to variations in hematocrit and the protein concentration of individual patient test samples and the tendency of analytes to bind to blood cells and proteins and thereby lead to inaccuracies in the determination of the amount of cyclosporine.

Various methods are known for extracting cyclosporine from blood. These methods involve the use of certain surfactants and organic solvents to release cyclosporine from its bound form. The prior art methods include a multi-step protocol for cell lysis, cyclosporine extraction, and pre-test removal of interfering protein and cellular components of the samples. Organic solvents, primarily lower alcohols, are usually important active components of the known extraction reagents. However, these alcohol based organic solvents are volatile and toxic, and make it more difficult to obtain accurate results in detecting the amount of cyclosporine in a patient.

The prior art methods for extracting cyclosporine from blood also use precipitating reagents to precipitate protein, and use centrifugation to separate the precipitated material from the solubilized cyclosporine, thereby making the extraction more complicated.

U.S. Pat. No. 5,135,875 to Meucci et al discloses the extraction of a hydrophobic analyte from a biological test sample by using a precipitating reagent to precipitate interfering proteins from the biological test sample. The precipitating reagent comprises a zinc salt, a straight or branch chained alcohol having 1 to 4 carbon atoms, and a glycol, a glycerol, or a combination of glycol and glycerol. The extraction of the hydrophobic analyte is preferably accomplished by centrifuging the treated test sample to separate the supernatant solution containing the desired analyte. The solubilization reagent in Meucci et al comprises a non-ionic detergent, alkoxy(polyethyleneoxypropyleneoxy)-isopropanol, and saponin.

U.S. Pat. No. 6,190,873 to Davalian et al discloses a method for measuring the amount of cyclosporine by contacting an aqueous solution of the biological test sample with cyclosporine-label conjugate, and antibodies capable of binding to the cyclosporine-label conjugate to form a detectable complex, and correlating the detectable complex with the amount of cyclosporine in the sample. The cyclosporine sample to be analyzed can be pretreated to lyse cells which may be present, precipitate proteins which may be present, and/or solubilize cyclosporine which may be present. The sample is preferably pretreated by contacting with an organic solvent, preferably an alcohol such as methanol.

### DESCRIPTION OF THE PREFERRED EMBODIMENTS

The present invention utilizes an extracting reagent to achieve quantitative detection of hydrophobic analytes from a biological test sample. The hydrophobic analytes can include steroids, and drugs. The biological test sample can include serum, plasma, whole blood, urine and spinal fluid. The hydrophobic analyte is preferably the drug cyclosporine, which is used primarily as an immunosuppressant. The biological test sample is preferably whole blood.

The hydrophobic structure of cyclosporine leads to the formation of its relatively stable complexes with various macromolecular and cellular elements in the blood, such as red cells, white cells and lipoproteins. This is due to the relatively low solubility of cyclosporine in water, approximately 0.04 mg/g. In aqueous media cyclosporine becomes easily adsorbed by the less polar components of the solution. The typical partition pattern of cyclosporine in blood is defined as follows: 40-50% bound to red blood cells, 30-40% bound to plasma proteins, and 10-20% bound to leukocytes.

The inventive extracting reagent is an effective and efficient means for extracting cyclosporine from the test sample in a single step without the need for an organic solvent, without the need for a precipitating agent and without the need for separating the cyclosporine from the patient blood sample, such as, by centrifugation and the like.

The contacting of the biological test sample with the cyclosporine-extracting reagent produces a homogeneous, solubilized cyclosporine test sample that is ready for analysis in a suitable automated immunoanalyzer, such as the ADVIA Centaur® automated immunoanalyzer (Bayer Corp.).

The cyclosporine-extracting reagent comprises an effective amount of an aqueous solution of a zwitterionic detergent and saponin.

Saponin is a potent hemolytic agent and is included in the extracting reagent to lyse erythrocytes, that is, the alteration, dissolution, or destruction of red blood cells in such a manner that hemoglobin is liberated into the medium in which the cells are suspended. Saponin also serves to solubilize cellular material and cyclosporine.

The concentration of saponin in the extracting reagent can vary from about 0.1 weight % to about 1 weight %, and preferably about 0.25 weight % to about 0.5 weight %.

The zwitterionic detergent is a physiologically moderate surfactant that is included in the extracting reagent to maintain the solubility of the test sample components, including cyclosporine.

The concentration of the zwitterionic detergent in the extracting reagent can vary from about 0.1 weight % to about 1 weight %, and preferably about 0.25 weight % to about 0.5 weight %.

Examples of zwitterionic detergents include 3-[N, N-dimethyl-(3-myristoylaminopropyl)ammonio]-propanesulfate,

3-(4-heptyl)phenyl 3-hydroxy propyl)dimethylammonio-propane sulfate,

3-(decyldimethylammonio)propanesulfonate inner salt,

3-(N, N-dimethylmyristylammonio)propanesulfonate,

3-(N, N-dimethyloctadecylammonio)propanesulfonate,

3-(N, N-dimethyloctylammonio)propanesulfonate inner salt,

3-(N, N-dimethylpalmitylammonio)propanesulfonate,

3-(dodecyldimethylammonio)propanesulfonate inner salt, and other equivalent zwitterionic detergents.

The preferred zwitterionic detergent is N-dodecyl-N,N-dimethyl-3-ammonio-1-propanesulfonate, available under the trade names Zwittergent® 3-12 (Calbiochem) and Bioplus SB-12™ (Bioworld).

The extracting reagent can also include a viscosity maintaining compound in sufficient amounts to provide for more efficient aspiration of the cyclosporine test samples by the automated immunoanalyzer. Suitable viscosity maintaining compounds include sucrose or glycerin and other equivalents in effective amounts that can vary from the minimum amount above 0 to about 60 weight %, and preferably about 5 weight % to about 50 weight % of the extracting reagent.

The extracted, solubilized cyclosporine test sample can then be introduced into a suitable automated immunoanalyzer, such as the ADVIA Centaur® (Bayer Corp.) for detection and analysis without any special pretreatment to separate components such as precipitation and/or centrifugation.

The detection sample submitted for analysis contains all the blood components of the patient blood sample and all the components of the extracting reagent in a solubilized, homogeneous state. The cyclosporine in the patient blood sample can then be quantitatively analyzed and measured using a suitable automated immunoassay system, such as the ADVIA Centaur® automated immunoanalyzer (Bayer Corporation) or equivalent.

Thus, the inventive composition and method does not involve physical separation of the cyclosporine from the patient blood sample before the automated analytical test for cyclosporine is conducted, and is a simplification and improvement over the prior art.

The extraction reagent for cyclosporine from the patient blood sample accomplishes in a single operation the osmotic shock necessary to disrupt the red blood cells and facilitate the separation of bound cyclosporine from the complexes it has formed in the blood, cell lysis to homogenize the sample, and the solubilization of the entire patient blood sample to produce an extracted, solubilized cyclosporine released from the plasma components and cellular material in the patient blood sample, in an immunologically detectable form for quantitative detection of the cyclosporine analyte using cyclosporine-specific antibodies. If the analysis of cyclosporine was conducted without the extraction step it would show significant under-recovery.

The detection and analysis of cyclosporine performed in the automated immunoanalyzer is based on the reaction between the cyclosporine analyte from the blood sample and an appropriate cyclosporine specific antibody or binding agent for the cyclosporine analyte.

The preferred method of analysis is based on the competitive reaction of cyclosporine from the sample and a constant amount of pre-labeled cyclosporine tracer for the cyclosporine-specific antibody labeled with biotin.

The analyte is the cyclosporine molecule that is introduced to the patient blood through the administration of cyclosporine as a drug. The cyclosporine specific antibody is an important component of the automated immunoanalyzer's detection reagent composition.

Another important component of the immunoanalyzer detection reagent is a synthetic cyclosporine derivative labeled with a tracer molecule that can be quantitatively measured by the immunoanalyzer. The tracer is an acridinium ester. The detection method is based on measuring its luminescence using a photon counting photomultiplier that is known to those skilled in the art.

Thus, the cyclosporine specific antibody reacts in the automated immunoanalyzer with the cyclosporine analyte extracted from the patient blood sample and the tracer-labeled cyclosporine.

The analyte present in the test sample and the tracer compound compete for a limited number of binding sites, resulting in the formation of analyte-antibody complexes and tracer compound-antibody complexes. By maintaining a constant concentration of the tracer compound and the antibody, the ratio of the formation of analyte-antibody complex to tracer-antibody complex is directly proportional to the amount of analyte present in the test sample.

The cyclosporine-antibody reaction, or analyte-binding agent reaction requires access of the antigen recognizing domain of the cyclosporine specific antibody to the epitope fragment of the cyclosporine molecule, or analyte. The availability of this epitope region on the cyclosporine from the patient blood sample for the reaction is maintained by keeping the cyclosporine in solution and is not hindered by the presence of lipoproteins and other macromolecular components in the extracted cyclosporine test sample.

The cyclosporine analyte-antibody complexes and tracer antibody complexes are captured by a paramagnetic solid phase reagent, for example, paramagnetic microparticles coated with streptavidin in a ratio defined by analyte concentration in the test sample and quantified using a chemiluminescent detection system by comparison with a standard calibration curve that reveals the cyclosporine concentration.

The paramagnetic solid phase reagent is another important component of the automated immunoanalyzer detection reagent composition. It comprises paramagnetic microparticles chemically coated with streptavidin. Molecules of streptavidin have a strong and selective natural specificity to biotin and are widely used to react with biotin-labeled molecules, such as the cyclosporine-specific antibody in the immunoassay.

The method of the present invention relies on the reaction between streptavidin-coated paramagnetic particles and the biotin-labeled cyclosporine specific antibody. This reaction immobilizes the cyclosporine specific antibody and allows the capture of both cyclosporine from the sample and the acridinium ester labeled cyclosporine of the detection reagent.

Higher amounts of cyclosporine in the tested sample translate to a smaller number of the detection reagent's tracer-cyclosporine molecules bound to the antibody due to the competition of these two forms of cyclosporine.

Therefore, the measured analytical signal is inversely proportional to the concentration of cyclosporine in the patient blood sample subjected to analysis in the automated immunoanalyzer.

The final form of the detection sample submitted to the automated immunoanalyzer for analysis comprises the solubilized patient blood sample containing the cyclosporine for analysis, and the extracting reagent in a fixed volumetric ratio varying from about 1:1 to about 1:9, respectively, and preferably about 1:2 to about 1:4, respectively.

An important advantage in using the extracting reagent of the present invention is that no organic solvents are involved, no precipitation step is involved, and no centrifugation step is required. Treatment of the patient blood sample with the extracting reagent produces a homogeneous, extracted, solubilized cyclosporine test sample wherein the released cyclosporine can be quantitatively recognized by the labeled biotin antibody.

### EXAMPLE 1

Eight patients were dosed with a therapeutic amount of cyclospoprine in a clinical setting. Blood sample aliquots of about 1-2 milliliters were then drawn from each patient about 24 hours after administering the cyclosporine. Each blood sample was treated with the inventive extracting reagent in a volumetric ratio of blood sample to extracting reagent of 1:4 respectively.

The extracting reagent consisted of 0.3125 weight % N-dodecyl-N,N-dimethyl-3-ammonio-1-propanesulfonate (Bioplus SB-12™-Bioworld), 0.625 weight % saponin, 43.75 weight % glycerin, and the remainder, distilled water. The extracting reagent extracted and solubilized the cyclosporine in each patient blood sample to produce a homogeneous blood sample with the extracted cyclosporine.

20 microliters of each homogeneous blood sample was then introduced into and analyzed in an ADVIA Centaur (Bayer Corp.) automated immunoanalyzer. The results are tabulated in Table 1.

Referring to Table 1, the term “unspiked” refers to the amount of cyclosporine detected in each patient blood sample 24 hours after each patient was dosed with a therapeutic amount of cyclosporine. The term “spiked” refers to the addition of a known gravimetrically measured amount of cyclosporine to the patient blood sample approximately six months later.

A 1 ml blood sample was also taken from a non-transplant “control” donor who was not undergoing immunosuppressant therapy. The control donor was given an initial measured 100 ng/ml dose of cyclosporine to mimic the patient sample. The analysis of the cyclosporine from the non-transplant control donor appears in Table 1 as “control patient”. The analysis of cyclosporine from the non-transplant control donor served as a control to assess the accuracy of subsequent spiking with additional measured doses of cyclosporine.

Each patient blood sample, was stored at −80° C. for approximately six months. Each blood sample was then thawed to ambient temperature, spiked with 200 ng/ml of cyclosporine and incubated for 18 hours at 4° C. A 20 microliter aliquot from each spiked sample was tested by the Bayer Corp. ADVIA Centaur® immunoanalyzer.

The data tabulated in Table 1 under the heading “spiked 1^{st} rep” represents the analysis of the total amount of cyclosporine after the patient blood sample was spiked with 200 ng/ml of cyclosporine. The data for the 1^{st} rep and 2^{nd} rep in Table 1 represent two measurements of the same sample in the same run that were done at the same time. Two reps were taken and analyzed to show reproducibility and to make the results more statistically significant.

The percent recovery of the cyclosporine was then calculated. Both reps were used to compute the percent recovery based on an average of the 1^{st} and 2^{nd} reps with the results tabulated in the “% Recovery” column of Table 1.

The results in Table 1 demonstrate the effectiveness of the inventive method for extracting cyclosporine. Comparison of the analytically recovered and measured amounts of cyclosporine with the gravimetrically measured spiked amounts of cyclosporine provided an indication of the accuracy of the method and enabled the efficiency of the cyclosporine extraction from the blood to be evaluated.

The accuracy of the method and the extraction in this experiment was judged by comparing expected recovery which is 200 ng/ml with the actual measured recovered dose. Ideally, the % recovery should be 100%, but in actuality there was a ±10% deviation, which is well within the acceptable limitations for this analysis.

TABLE 1

| | | Spiked | Spiked | % Recovery of |
|---|---|---|---|---|
| | Unspiked | 1st rep | 2nd rep | Spiked |
| Sample | (ng/ml) | (ng/ml) | (ng/ml) | Cyclosporine |
| patient sample 1 | 61.1 | 278.6 | 265.3 | 105.4 |
| patient sample 2 | 160.8 | 331.4 | 332.0 | 85.4 |
| patient sample 3 | 41.0 | 263.3 | 250.7 | 108.0 |
| patient sample 4 | 42.7 | 269.1 | 255.0 | 109.7 |
| patient sample 5 | 289.8 | 473.0 | 483.2 | 94.2 |
| patient sample 6 | 148.0 | 324.6 | 335.7 | 91.1 |
| patient sample 7 | 108.3 | 298.6 | 281.9 | 91.0 |
| patient sample 8 | 77.3 | 288.6 | 272.8 | 101.7 |
| control patient | 112.6 | 315.8 | 325.1 | 103.9 |

What is claimed is:

1. A reagent composition for extracting a hydrophobic analyte from a heterogeneous biological test sample, without an organic solvent and without a precipitating agent, comprising an aqueous solution of:
(a) a zwitterionic detergent; and
(b) saponin,
wherein each of the components are present in sufficient amounts that are effective to release the hydrophobic analyte from the biological test sample in a homogenous solution.

2. The reagent composition of claim 1, wherein the amount of zwitterionic detergent varies from about 0.1 weight % to about 1.0 weight %.

3. The reagent composition of claim 1, wherein the amount of saponin varies from about 0.1 weight % to about 1 weight %.

4. The reagent composition of claim 1, wherein the biological test sample is selected from the group consisting of serum, plasma, whole blood, urine, and spinal fluid.

5. The reagent composition of claim 1, wherein the biological test sample is whole blood.

6. The reagent composition of claim 1, wherein the hydrophobic analytes are selected from the group consisting of steroids and drugs.

7. The reagent composition of claim 1, wherein the hydrophobic analyte is cyclosporine.

8. The reagent composition of claim 1, wherein zwitterionic detergent is selected from the group consisting of
3-[N, N-dimethyl(3-myristoylaminopropyl)ammonio]-propanesulfate,
3-(4-heptyl)phenyl 3-hydroxy propyl)dimethylammonio propane sulfate,
3-(decyldimethylammonio)propanesulfonate inner salt,
3-(N, N-dimethylmyristylammonio)propanesulfonate,
3-(N, N-dimethyloctadecylammonio)propanesulfonate,
3-(N, N-dimethyloctylammonio)propanesulfonate inner salt,
3-(N, N-dimethylpalmitylammonio)propanesulfonate, and
3-(dodecyldimethylammonio)propanesulfonate inner salt.

9. The reagent composition of claim 1, also including at least one viscosity additive selected from the group consisting of glycerin and sucrose.

10. The reagent composition of claim 8, wherein the amount of viscosity additive varies from about 5 weight % to about 50 weight %.

11. An immunoassay method for determining the amount of a hydrophobic analyte in a biological test sample, comprising:
(a) contacting the test sample with an effective amount of extracting reagent without an organic solvent and without a precipitating agent comprising:
(i) a zwitterionic detergent; and
(ii) saponin,
wherein each of the components of the extracting agent are present in amounts effective to release the hydrophobic analyte in a homogeneous solution without centrifugation;
(b) introducing the homogeneous solution containing the hydrophobic analyte into an automated iummunoanalyzer, and
(c) detecting the amount of hydrophobic analyte with the automated immunoanalyzer.

12. The method of claim 11, wherein the amount of zwitterionic detergent varies from about 0.1% to about 1.0%.

13. The method of claim 11, wherein the amount of saponin varies from about 0.1% to about 1%.

14. The method of claim 11, wherein the extraction of the analyte from the test sample is done in a single step.

15. The method of claim 11, wherein the biological test sample is selected from the group consisting of serum, plasma, whole blood, urine, and spinal fluid.

16. The method of claim 11, wherein the biological test sample is whole blood.

17. The method of claim 11, wherein the hydrophobic analyte is selected from the group consisting of steroids and drugs.

18. The method of claim 11, wherein the hydrophobic analyte is cyclosporine.

19. The method of claim 11, wherein the extracting reagent includes at least one viscosity additive selected from the group consisting of glycerin and sucrose.

20. The method of claim 11, wherein the amount of viscosity additive varies from about 5 weight % to about 50 weight %.
